(19) United States
(12) Patent Application Publication
(10) Pub. No.: US 2022/0334109 A1
(43) Pub. Date: Oct. 20, 2022

(54) DEVICE AND METHOD FOR DETECTING INFLAMMATION

(71) Applicant: INNOTECH PRECISION MEDICINE, INC., Boston, MA (US)

(72) Inventors: Roya Khosravi-FAR, Boston, MA (US); Ramin M. Hakami, Bristow, VA (US)

(21) Appl. No.: 17/702,107

(22) Filed: Mar. 23, 2022

Related U.S. Application Data
(60) Provisional application No. 63/165,372, filed on Mar. 24, 2021.

(51) Int. Cl. G01N 33/543; G01N 33/68

(52) U.S. Cl. CPC G01N 33/5438; G01N 33/6893; G01N 33/6869; G01N 2333/5412; G01N 2333/5421; G01N 2800/52; G01N 2333/5428; G01N 2333/521

(57) ABSTRACT

In one aspect, a system for detecting inflammation is disclosed, which includes at least one sensor comprising at least one port for receiving a biological sample, and at least one electrochemical cell in fluid communication with said at least one port for receiving said biological sample, said electrochemical cell comprising at least two electrically conductive electrodes, where at least one of said electrodes is functionalized with at least one probe exhibiting specific binding to at least one inflammation biomarker. The system may further include a circuitry for detecting a redox current flowing through said at least one electrode and/or an electrical impedance across said electrodes in response to interaction of the functionalized electrode with the sample, and generating signals in response to said detection. By way of example, the biological sample can be a subject's blood serum.

### RELATED APPLICATION

The present patent application claims priority to U.S. provisional patent application No. 63/165,372 filed on Mar. 24, 2021, which is titled “Device and Method for Detecting Inflammation”, and which is incorporated herein by reference in its entirety.

The present patent application also incorporates by reference, in their entireties, the following U.S. patent applications: U.S. provisional patent application No. 63/082,694 filed on Sep. 24, 2020 and U.S. provisional patent application No. 63/143,690 filed on Jan. 29, 2021, each of which is titled “Device and Method for Detecting Pathogens,” and U.S. patent application Ser. No. 17/484,842 filed on Sep. 24, 2021, which is titled “Systems, Apparatus, and Methods for Detecting Pathogens.”

### SEQUENCE LISTING

The instant application contains a Sequence Listing which has been submitted electronically in ASCII format and is hereby incorporated by reference in its entirety. Said ASCII copy, created on Jun. 27, 2022, is named 1892254-0002-003-101_SL.txt and is 4,355 bytes in size.

### BACKGROUND

The present teachings are generally directed to systems and methods for detecting an inflammatory response, e.g., an inflammatory response exhibited by a patient infected with a pathogenic agent or as a result of a side effect to cancer immunotherapy, or tissue damage. The response of a patient's immune system to a pathogenic infection or other pathologic and unregulated elevation of cytokines can result in the release of cytokines and/or chemokines and/or other biomarkers of inflammation leading to organ damage and elevation of organ damage markers. As an example, interleukin-6 (IL-6) is released from different cells to initiate and maintain antiviral response. In fact, interleukin-6 (IL-6) is one of the main mediators of inflammatory and immune response initiated by infection or injury. For example, increased levels of IL-6 are found in many patients with severe COVID-19, influenza, and bacterial infection. As an example, in response to SARS-CoV-2 infection. IL-6 concentrations in plasma can dramatically increase from picograms/ml to micrograms/ml.

Very high Levels of IL-6 (above 25 pg/ml, with reference range of <or =1.8 pg/mL) seem to be associated with an inflammatory response, respiratory failure, the need for mechanical ventilation and/or intubation and mortality in COVID-19 patients. In a meta-analysis including nine studies (total of 1426 patients) reporting on IL-6 and outcome in COVID-19, mean IL-6 levels were more than three times higher in patients with complicated COVID-19 compared with those with non-complicated disease, and IL-6 levels were associated with mortality risk.

Elevated serum C-reactive protein (CRP) is also a biomarker of severe clinical manifestation of infectious diseases, including COVID-19. Expression of CRP is driven by IL-6. Other markers, such as ferritin, D-dimer, aspartate aminotransferase, procalcitonin and creatinine, that are generally elevated in response to organ damage, which is a consequence of cytokine storm, are also elevated in response to cytokine storm. Further. CRP can indicate tissue damage. e.g., cardiac tissue damage.

Sepsis is a life-threatening condition that is a result of a dysregulated host response to infection. This can result in tissue damage and multiple organ dysfunction. IL-6 and CRP are significantly upregulated in sepsis. Another key marker for sepsis is procalcitonin (PCT). PCT levels of >2.0 μg/L indicate a high probability of systemic bacterial infection, with a risk for progression to sepsis or septic shock. PCT levels of <0.5 μg/L indicate a low likelihood of systemic bacterial infection, with low risk of progression to sepsis or septic shock. Accordingly, there is a need for systems and methods that can efficiently and rapidly detect and monitor a dysregulated inflammatory response in a patient. Such rapid detection allow interventions such as FDA-approved anti-inflammatory medication and targeted therapies to be applied promptly. For example, there are 2 classes of Food and Drug Administration (FDA)-approved IL-6 inhibitors. Anti-IL-6 receptor monoclonal antibodies (mAbs) (i.e., sarilumab, tocilizumab) and anti-IL-6 mAbs (i.e., siltuximab) are available for patients with systemic inflammation.

### SUMMARY

In one aspect, a system for detecting inflammation is disclosed, which includes at least one sensor comprising at least one port for receiving a biological sample, e.g., a liquid biopsy sample, such as a blood, a urine or a saliva sample, and at least one electrochemical cell in fluid communication with said at least one port for receiving said biological sample, said electrochemical cell comprising at least two electrically conductive electrodes. At least one of said electrodes is functionalized with at least one molecular recognition probe exhibiting specific binding to at least one inflammatory target biomarker (such as inflammation biomarker, tissue-damage biomarker), or genetic component associated with the inflammatory target biomarker or the tissue-damage biomarker. The system can further include circuitry for detecting a redox current flowing through said at least one electrode and/or an electrical impedance across said electrodes in response to interaction of the functionalized electrode with the sample, and generating signals in response to said detection. By way of example, the biological sample can be a subject's blood serum.

In some embodiments, the system can include a plurality of electrochemical cells, each of which is configured for detection of a different inflammation biomarker, such as the inflammation biomarkers disclosed herein. The inflammation biomarker can be multiplexed with relevant biomarkers for organ damage. For example, IL-6 sensors can be combined with ferritin and also other inflammatory markers such as C-reactive protein (CRP). In some embodiments, at least one of the electrochemical cells is configured for detection of an inflammation biomarker and at least another one of the electrochemical cells is configured for detection of a genetic component coding for that inflammation biomarker. In some other embodiments, at least one of the electrochemical cells is configured for detection of an inflammation biomarker and at least another one of the electrochemical cells is configured for the detection of a tissue-damage biomarker that is different from the inflammation biomarker. In another embodiment, the system can include electrochemical cells that are configured for detection of an inflammation biomarker, a tissue-damage biomarker, and genetic component(s) coding for the inflammation biomarker and/or the tissue-damage biomarker.

In some embodiments, the probe can be any of an aptamer, a SOMAmer, an antibody and/or a raptomer, a megastar or any combination thereof. Levels of these biomarkers can also be detected from their gene expression level by quantifying their mRNA level in liquid biopsy (blood, serum) or in exosomes that may have higher levels of these inflammatory and organ damage factors.

The system can further include an analyzer that is in communication with the circuitry for receiving the signals generated by the circuitry and processing those signals to determine the level of the inflammation biomarker(s) and/or tissue-damage biomarker(s) in the biological sample. In some embodiments, the analyzer can further be configured to determine, based on the detected level of one or more inflammation biomarker(s), the onset and/or occurrence of a cytokine storm.

In some embodiments, the system can include a plurality of electrochemical cells with a first electrochemical cell functionalized with a molecular recognition probe exhibiting specific binding to the inflammation biomarker and/or the tissue-damage biomarker and a second electrochemical cell functionalized with a molecular recognition probe exhibiting specific binding to a genetic component associated with the inflammation biomarker or the tissue-damage biomarker. The system can further include an analyzer configured to receive the detection signals generated by said first and second electrochemical cells and process those detection signals to determine whether the inflammation biomarker and/or the tissue-damage biomarker are present in the biological sample. By way of example, in some such embodiments, the analyzer is configured to indicate the presence of the inflammation biomarker or the tissue-damage biomarker in the biological sample when both of the detection signals generated by the first and second electrochemical cells are positive signals (i.e., both signals indicate that the inflammation biomarker or the tissue-damage biomarker is present in the sample). Further, the analyzer can be configured to indicate the target biomarker is not present in the sample at a level above the sensor's limit-of-detection when both detection signals are negative (i.e., neither signal indicates the presence of the biomarker in the biological sample). The analyzer can indicate a non-conclusive result when one of the detection signals generated by the first and the second electrochemical cells is positive and the other detection signal is negative.

The inflammation biomarker can be a chemokine or a cytokine. Some examples of such inflammation biomarkers include, without limitation, interferon-γ, interleukin-6, interleukin-8, interleukin-10, interleukin-18 and soluble interleukin-2 receptor alpha, CXCL9 and CXCL10. A variety of tissue-damage biomarkers, e.g., C-reactive protein, can also be detected using a sensor according to the present teachings. In some cases, an inflammation biomarker can also function as a tissue-damage biomarker (e.g., CRP).

In a related aspect, a method for detecting an inflammation biomarker and/or a tissue-damage biomarker in a biological sample is disclosed, which comprises introducing the biological sample into an electrochemical cell of a sensor having at least one working electrode and a reference electrode, wherein the working electrode is functionalized with at least one molecular recognition probe exhibiting specific binding to said inflammation biomarker, said tissue-damage biomarker, or at least one genetic component coding for that inflammation biomarker or the tissue-damage biomarker, and measuring a redox current flowing through said working electrode or an electrical impedance across said working electrode and said reference electrode in response to interaction of said functionalized electrode with the sample so as to generate detection signals.

The detection signals are processed to determine whether the inflammation biomarker and/or the tissue-damage biomarker are present in the biological sample at a concentration level above a limit-of-detection of the sensor.

In some embodiments, a sensor according to an embodiment for the detection of target biomarkers can include an electrochemical analysis cell having a working electrode (herein also referred to as a sensing electrode), a counter electrode and a reference electrode. The working electrode can be functionalized in a manner discussed herein to configure the electrochemical analysis cell for detection of one or more proteins or one or more RNA and/or DNA segments associated with a target biomarker. More specifically, at least one aptamer or at least one oligonucleotide can be coupled to the working electrode, where said at least one aptamer is configured to specifically bind to at least one protein associated with a target biomarker and said at least one oligonucleotide comprises a nucleotide sequence that is complementary to a nucleotide sequence of at least one RNA or DNA segment of that biomarker or another biomarker.

In some embodiments, a plurality of carbon nanotubes are disposed on the working electrode, where the plurality of carbon nanotubes are functionalized with at least one antibody, aptamer or other molecular identification element configured to specifically bind to at least one protein associated with an inflammatory target biomarker or functionalized with at least one oligonucleotide having a nucleotide sequence complementary to nucleotide sequence of at least one RNA or DNA segment of that biomarker or another biomarker.

In some embodiments, a sequence-specific oligonucleotide or a protein-specific aptamer will be anchored to SWCNT via oligonucleotide DNA anchors. As an example, 8-15 AT (adenine, thymine) repeats can be used as DNA anchors for the attachment of oligonucleotide, for recognition of specific sequences in the genetic component (e.g., mRNA) of a gene coding for an inflammatory target biomarker, or aptamer, for recognition of specific proteins of a biomarker, to SWCNTs.

In some embodiments, nucleic acid spacers or linkers such as spacer 18 also known as HEG spacer (hexaethylene glycol) can be placed at 5′ or 3′ of the oligonucleotide or the aptamer for coupling to the SWCNT. In addition to single HEG spacer, multiple repeats of HEG spacers, for example, 3 or 5, can be used for configuration of the optimal positioning and optimal presentation of the oligonucleotide or aptamer for the recognition and capture of their target sequence or target protein.

The electrochemical analysis cell generates a detection signal when at least a protein or at least an RNA or DNA segment associated with a gene coding for a biomarker, when present in the sample, binds to at least one of the aptamers or the oligonucleotides, respectively, associated with the working electrode. In some embodiments, a plurality of different types of aptamers can be coupled to the working electrode, where the aptamers are configured to bind to different proteins associated with different inflammatory target biomarkers. Further, in some embodiments, a plurality of different types of oligonucleotides can be coupled to the working electrode, where the different types of oligonucleotides have complementary sequences to different RNA or DNA segments of a gene coding for a target biomarker.

The at least one electrochemical cell of a sensor according to the present teachings can be housed in a housing, which can be formed, for example, of a polymeric material. For example, PDMS (polydimethylsiloxane) can be employed for fabricating the housing.

In the above sensor, the electrochemical cell generates a detection signal when said at least one protein or said at least one RNA or DNA segment associated with the target biomarker, when present in the sample, binds to at least one of said aptamers or said oligonucleotide, respectively.

The sensor can further include an analysis module that is in communication with the electrochemical analysis cell to receive the detection signals generated by the electrochemical cell and process those detection signals to determine whether a target pathogen is present in the sample. For example, in some embodiments, when detection signals generated by both the protein-detecting sensing unit and the RNA/DNA-detecting sensing unit exceed certain predefined thresholds, the analyzer can indicate that the target biomarker is present in the sample.

In some embodiments, the sensor can be disposed within a disposable cartridge.

In some embodiments, the sensor can include a plurality of sensing units, where one of said sensing units comprises aptamer-functionalized carbon nanotubes and another one of said sensing units comprises oligonucleotide-functionalized carbon nanotubes. In some such embodiments, the carbon nanotubes are functionalized with a plurality of aptamers that exhibit specific binding to different inflammatory target biomarkers.

In some embodiments, a sensor includes a first sensing unit that can include at least one sensing module in the form of an electrochemical sensor having a sensing electrode that is functionalized with at least one affinity binding element that exhibits specific binding to at least one protein associated with an inflammatory target biomarker. The sensing electrode can be functionalized with a variety of different affinity binding elements. For example, the affinity binding elements can be an aptamer, an oligonucleotide, a morpholino, and/or an affirmer, though any suitable affinity binding element that can exhibit specific binding to a protein of interest can be employed. In some embodiments, such an affinity binding element can exhibit a binding affinity in low nanomolar range (10-9) to picomolar range (10-12).

In some embodiments, the first sensing unit can include a plurality of sensing modules where the sensing modules are functionalized with different molecular recognition elements (herein also referred to as affinity binding elements) such that each of the sensing modules is capable of detecting a different protein associated with one or multiple inflammatory target biomarkers of interest. In this manner, a multiplexed sensing unit can be fabricated, which allows concurrent detection of multiple proteins associated with multiple inflammatory target biomarkers.

In some embodiments, the sensor can further include a second sensing unit that can include at least one sensing module having an electrochemical sensor having a sensing electrode functionalized with at least one affinity binding element exhibiting specific binding to at least one genetic component associated with the inflammatory target biomarker, e.g., an RNA and/or a DNA segment. By way of example, such an affinity binding element can be an oligonucleotide having a nucleotide sequence that is complementary to the nucleotide sequence of the target genetic component associated with an inflammatory target biomarker of interest.

In another aspect, a sensor for detecting an inflammatory target biomarker is disclosed, which includes at least one sensing unit comprising a sensing electrode that is functionalized with at least a first affinity binding element exhibiting specific binding to a protein biomarker or a genetic component of a gene encoding for that biomarker such that a binding of the biomarker or its associated genetic element (e.g., mRNA) to said affinity binding element changes at least one electrical property of the sensing electrode, and at least another sensing unit that comprises a surface for performing surface enhanced Raman spectroscopy (SERS), where the SERS surface is functionalized with at least an affinity binding element exhibiting specific binding to the biomarker of interest for obtaining a Raman signal associated with any of said biomarker and said affinity binding element in response to binding of the biomarker to said functionalized SERS surface. In some such embodiments, the electrochemical and the Raman sensing are achieved using a single electrode that is a working electrode of an electrochemical sensor and is also configured as a SERS substrate.

In some embodiments, the first and the second affinity binding elements are the same, while in other embodiments, the first affinity binding element can be different from the second affinity binding element.

The sensor can further include a laser (e.g., a diode laser) that can generate radiation that is suitable for exciting at least one Raman active mode of any of the biomarker and the affinity binding element. The sensor can further include a photodetector for detecting Raman scattered radiation generated in response to the excitation of said at least one Raman active mode and generating at least one Raman detection signal.

The sensor can also include another detector (herein referred to for ease of description as an “electrical detector”) that is in communication with the sensing electrode for measuring said electrical property of the sensing electrode and generating a detection signal indicative of said measured property (e.g., a change in the electrical resistance of the sensing electrode).

The sensor can also include an analyzer in communication with said photodetector and said electrical detector for receiving the Raman as well as the electrical detection signal and processing those signals to determine whether any of the Raman detection signal and the electrical detection signal is indicative of the specific binding of an inflammatory biomarker to the affinity binding element. In some embodiments, the analyzer is configured to indicate the presence of a target inflammatory biomarker in a sample under investigation when both the Raman and the electrical signals indicate the presence of the target biomarker in the sample.

Further understanding of various aspects of the present teachings can be obtained by reference to the followed detailed description in conjunction with the associated drawings, which are described briefly below.

### BRIEF DESCRIPTION OF THE DRAWINGS

FIG. 1A schematically depicts a system according to an embodiment of the present teachings for detecting a plurality of inflammation biomarkers in a blood sample,

FIG. 1B schematically depicts a sensing module of the system depicted in FIG. 1A,

FIGS. 2A and 2B provide examples of oligonucleotide sequences of aptamers that are suitable for specific binding to several cytokines and other inflammation biomarkers. FIG. 2A discloses SEQ ID NOS 1-11, respectively, in order of appearance. FIG. 2B discloses SEQ ID NOS 12-16, respectively, in order of appearance.

FIG. 3A schematically depicts an electrochemical analysis cell according to an embodiment in which the working electrode of the cell is functionalized with a plurality of oligonucleotide probes,

FIG. 3B schematically depicts an electrochemical analysis cell according to an embodiment in which the working electrode of the electrochemical analysis cell is functionalized with a plurality of aptamer probes,

FIG. 4A schematically depicts an electrochemical analysis cell according to an embodiment in which the working electrode of the electrochemical analysis cell is functionalized with a plurality of carbon nanotubes, which are in turn functionalized with a plurality of aptamers,

FIG. 4B schematically depicts an electrochemical analysis cell according to an embodiment in which the working electrode of the cell is functionalized with a plurality of carbon nanotubes, which are in turn functionalized with a plurality of oligonucleotide probes,

FIG. 5A schematically depicts an electrochemical cell comprising two interdigitated electrodes, where one of the electrodes is functionalized with a plurality of aptamer probes,

FIG. 5B schematically depicts an electrochemical cell comprising two interdigitated electrodes, where one of the electrodes is functionalized with a plurality of oligonucleotide probes,

FIG. 6 is a partial schematic view of an electrochemical cell in which a heating element is disposed underneath the cell's working electrode for heating thereof, and

FIG. 7A schematically depicts an analysis module for processing data generated by a sensor according to the present teachings,

FIG. 7B schematically depicts an example of the implementation of the analysis module depicted in FIG. 7A,

FIG. 8 schematically depicts a sensing electrode that functions both as an electrical sensing electrode as well as a Raman sensing electrode, and

FIG. 9 schematically depicts a SERS module suitable for use in some embodiments of the present teachings.

### DETAILED DESCRIPTION

The present disclosure is generally directed to systems and methods for detection of inflammation biomarkers and tissue-damage biomarkers, which are herein collectively referred to as a “a target biomarker” or “an inflammatory target biomarker” in a biological sample, e.g., a blood or urine sample.

Various terms are used herein in accordance with their ordinary meanings in the art. For example, the term “aptamer” refers a nucleotide polymer with a specific affinity for a particular target molecule.

The term “nanoparticle,” as used herein, refers to a material structure having a maximum dimensional size (e.g., a diameter or other cross-dimensional size) that is equal to or less than about 1 micron, e.g., in a range of about 100 nanometers to about 500 nanometer, or in a range of about 200 nanometers to about 600 nanometers, or in a range of about 300 nanometers to about 700 nanometers, or in a range of about 400 nanometers to about 800 nanometers.

The term “affinity binding element,” as used herein, refers to a material structure, e.g., a polymer, that can exhibit a specific binding to an analyte. As discussed in more detail below, some examples of affinity binding elements include, without limitation, aptamers and other oligonucleotides.

The term “substantially,” as used herein, refers to state or condition that differs, if any, from a complete state or condition by at most 10%.

The present teachings are generally directed to systems and methods for detection of one or more cytokines and/or chemokines and/or other biomarkers of inflammation (or organ damage), which is herein also referred to as “a target biomarker” in a biological sample obtained from a subject. In some such embodiments, the detection of the protein or nucleic acid of cytokines and/or chemokines can be achieved together with the detection of proteins and/or genetic components (RNA and/or DNA segments) of one or more pathogens that are likely to have induced the production of the cytokines and/or the chemokines, as discussed in more detail below. In other embodiments, the present teachings disclose a stand-alone system that is dedicated to the detection of one or more cytokines and/or chemokines in a sample, e.g., in a blood sample.

Innate, or nonspecific, immunity is the defense system that provides protection against all antigens. Pro-inflammatory cytokines such as interferon-γ, interleukin-6, interleukin-8, interleukin-10, interleukin-18 and soluble interleukin-2 receptor alpha (a marker of T-cell activation) are generally present. High elevated serum interleukin-6 levels are found in CAR T-cell therapy-induced cytokine storm and several other cytokine storm disorders. Additionally, CXCL9 and CXCL10, chemokines induced by interferon-γ are also present. Autopsy studies have also shown that some cases of lethal COVID-19 are associated with extensive multi-organ inflammation, often in the absence of the virus in those organs.

As an example, interleukin-6 (IL-6) is released from different cells to initiate and maintain antiviral response. In fact, interleukin-6 (IL-6) is one of the main mediators of inflammatory and immune response initiated by infection or injury. For example, increased levels of IL-6 are found in many patients with COVID-19. In response to SARS-CoV-2 infection, IL-6 concentrations in plasma can dramatically increase from picograms/ml to micrograms/ml.

Very high Levels of IL-6 (above 25 pg/ml, with reference range of <or =1.8 pg/mL) seem to be associated with an inflammatory response, respiratory failure, the need for mechanical ventilation and/or intubation and mortality in COVID-19 patients. In a meta-analysis including nine studies (total of 1426 patients) reporting on IL-6 and outcome in COVID-19, mean IL-6 levels were more than three times higher in patients with complicated COVID-19 compared with those with non-complicated disease, and IL-6 levels were associated with mortality risk.

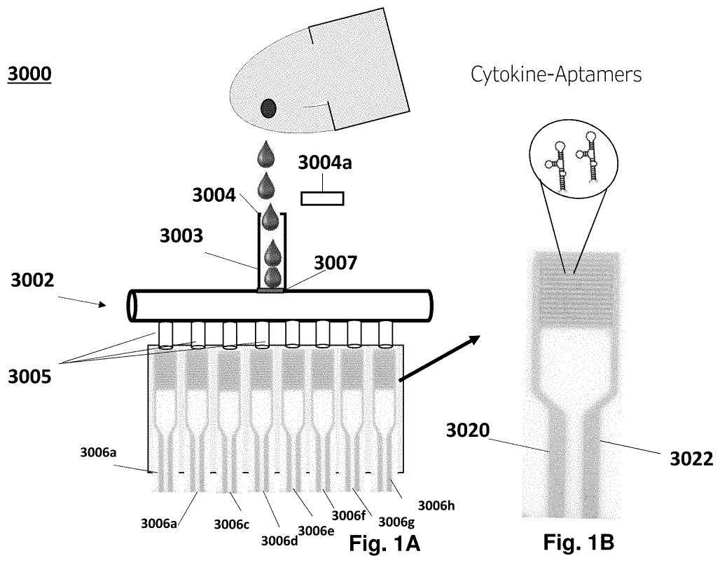

With reference to FIGS. 1A and 1B, a system 3000 according to an embodiment of the present teachings for detecting a plurality of inflammatory target biomarkers can include a sample collection unit 3002 for receiving a biological sample (e.g., liquid biopsy sample, such as a blood, a urine or a saliva sample) from a subject. e.g., via a finger prick or venipuncture. The following embodiments are described with reference to an example in which the biological sample is a blood sample. It should, however, be understood that the present teachings can be employed to analyze other liquid biopsy samples, such as urine and salvia samples.

In this embodiment, the sample collection unit includes a collection tube 3003 having an input port 3004 through which a blood sample can be introduced into the collection tube. A cap 3004a can then be coupled to the input port, via engaging a plurality of threads provided on the external surface of the collection tube in proximity of the input port with a plurality of internal threads of the cap, so as to close off the collection tube. When the biological sample is a blood sample, the blood can then be processed to separate plasma or serum, which will then be mixed with a protein processing buffer, which was previously introduced into the collection tube. e.g., by shaking or flipping the sample collection unit. In some cases, a change in the color of the capture buffer, e.g., from yellow to pink, can indicate that sufficient mixing of the blood sample with the protein processing buffer has been achieved. By way of example, protein processing buffer can be PBS (potassium-buffered saline) or HBSS (Hank's balanced salt solution) can be used as the buffer. Anti-microbial (e.g., gentamicin) and anti-fungal (e.g., amphotericin B) can be optionally added.

In this embodiment, once the mixing of the sample plasma/serum and the buffer has been achieved, a barrier 3007 positioned between the collection tube and a fluid distribution network 3005 can be opened to allow the mixture of the plasma/serum sample and the processing buffer to enter the fluid distribution network. In some embodiments, the barrier can be in the form of a frangible membrane, which can be broken by twisting the collection tube. In other embodiments, it can be an actuable valve, which can opened, e.g., via a mechanical switch or other suitable means.

The fluid distribution network includes a plurality of fluidic channels for delivering a portion of the plasma/serum sample to each of a plurality of sensing modules 3006a, 3006b, 3006c, 3006d. 3006e. 3006f. 3006g, and 3006h (herein collectively referred to as sensing modules 3006). Each of the sensing modules 3006 is configured to detect one inflammatory target biomarker, e.g., a cytokine and/or chemokine.

By way of example, in this embodiment, each of the sensing modules 3006 includes a pair of interdigitated gold electrodes, such as electrodes 3020 and 3022 shown in FIG. 1B. At least one of the electrodes (herein referred to as the sensing electrode) is functionalized with an antibody, aptamer. SOMAmer and/or raptomer 3025, which is configured to exhibit specific binding to a target inflammatory biomarker. The binding of a target inflammatory biomarker to the functionalized electrode can change an impedance across the sensing and the opposed reference electrodes, which can be measured and analyzed.

By way of example, in this embodiment, the sensing module 3006h is functionalized with an antibody, an aptamer and/or a SOMAmer and/or a raptomer and/or affimer and/or similar affinity binder to detect IL-6 (interleukin 6). Aptamers and SOMAmers suitable for specific binding to IL-6 are known and are commercially available. The binding of IL-6 to the functionalized sensing electrode can cause a change in an electrical impedance (e.g., DC or AC electrical resistance) measured across the electrode pair, thus allowing the detection of IL-6. The detection of impedance signals generated via interaction of IL-6 with the sensing module 3006h can be achieved in a similar manner as that discussed further below.

In some implementations of this sensor or other electrochemical sensors disclosed herein, a redox pair (e.g., Fe²⁺/Fe⁺³ such as that provided in a ferritin-iron system) can be employed and a redox current flowing through at least one functionalized electrode can be modulated in response to the coupling of an antigen to the functionalized electrode.

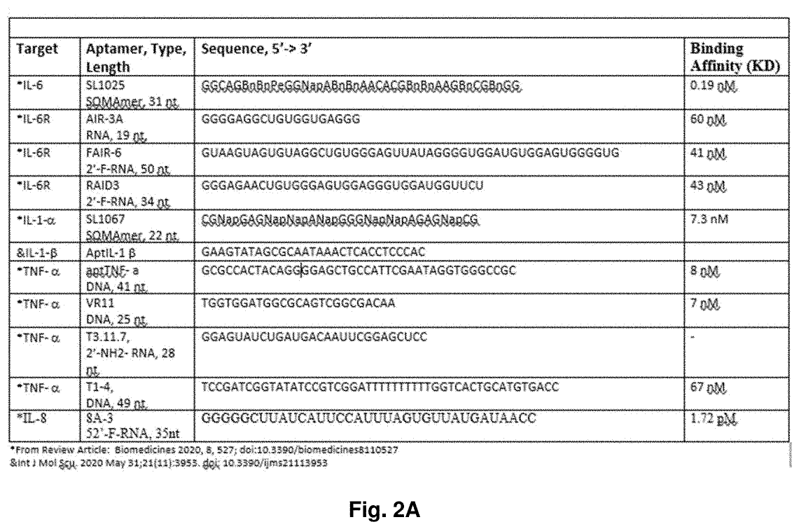

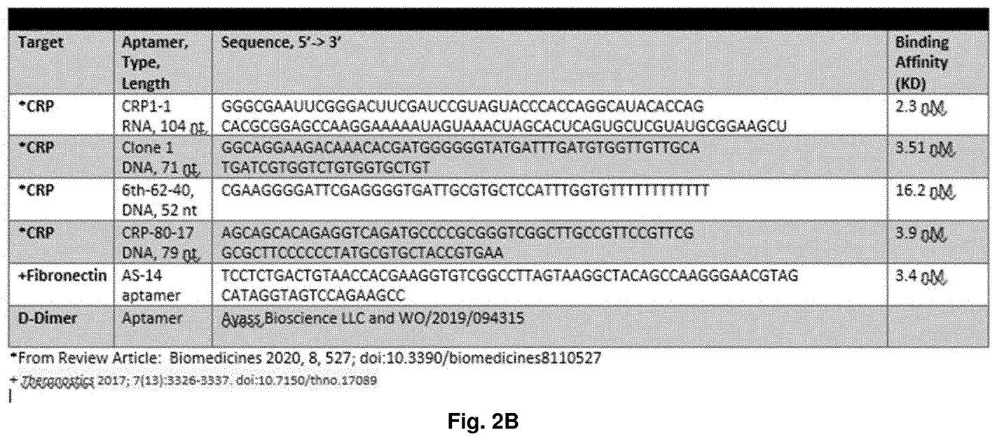

In this embodiment, the other sensing modules 3006 can be functionalized to detect inflammatory target biomarkers (such as cytokines and/or chemokines) other than IL-6. Some examples of such cytokines include, without limitation, IL-8, TNF-α. In addition, in some embodiments, one or more sensing modules of the system 3000 can be configured to detect one or inflammatory biomarkers, such as C-reactive protein (CRP), fibronectin, procalcitonin, and D-dimer. Some of these biomarkers, such as CRP, can also indicate tissue damage (e.g., elevated levels of CRP can be indicative of damage to cardiac tissue). Some examples of oligonucleotide sequences of aptamers that are suitable for specific binding to such cytokines and other inflammation biomarkers are presented in FIGS. 2A and 2B.

Moreover, in some embodiments, at least one of the sensing modules 3006 can be functionalized with an oligonucleotide having a nucleotide sequence that is complementary to the nucleotide sequence of at least one gene regulator, e.g., miRNA, that is indicative of inflammation. By way of example, several miRNAs (miR-132, miR-155, miR-31, miR-20b, miR222, miR192, miR-34a, let-7e, miR-193) that bind to 3′-UTR of a number of genes that regulate inflammation (snad3, ig/b, Foxo3, Runx1) or exacerbate inflammation (stat3, NkB, Ptgs2, Cend1, Thx21) are thought to be regulators of inflammatory response. In some embodiments, these cytokines can be detected in exosomes.

The detection of a panel of cytokines and/or chemokines and/or other biomarkers discussed above by the system 3000 can signal the onset and/or the occurrence of a cytokine storm and/or tissue damage. This information can in turn allow a medical professional to take the required steps for addressing the problem.

In some embodiments, the system 3000 can be employed in conjunction with a diagnostic device that is configured for detection of a pathogen. By way of example, if the diagnostic device indicates the presence of a pathogen, such as SARS-CoV-2 virus, in a sample obtained from a subject, the system 3000 can then be employed to identify the onset or the presence of a cytokine storm, if any, caused by the reaction of the subject's immune system to the pathogen.

The concurrent detection of one or more proteins, or one or more nucleic acids, or a combination of proteins and nucleic acids associated with a pathogen together with the detection of an inflammatory biomarker in manner described herein can enhance the detection of a pathogenic infection. Further, it helps expedite the measures needed to help the patient.

Cytokine Storm, Severity and Prognosis

Although the activation of innate immune response with elevation of cytokines/chemokines/inflammatory biomarkers constitutes a defense mechanism against pathogen infection, severe immune reaction in which the body releases too many cytokines/chemokines/inflammatory biomarkers into the blood too quickly can be harmful. A cytokine storm can occur as a result of an infection, autoimmune condition, or other diseases. Some diseases that may lead to the occurrence of a cytokine storm include multiple sclerosis (MS). Hemophagocytic lymphohistiocytosis (HLH), certain types of cancer, among others. It may also occur after treatment with some therapeutic modalities, e.g., certain types of immunotherapy. It has been reported as a major side effect in CAR T treatment. Signs and symptoms include high fever, inflammation (redness and swelling), and severe fatigue and nausea. Sometimes, a cytokine storm may be severe or life threatening and could lead to multiple organ failure and death.

A cytokine storm may result in elevation of IL-1-beta, IL-6, IL-5, IL-8, TNF-alpha, IFN-gamma, IP-10, MCP-1CCL3, GCSF. For patients that are hospitalized or in a clinical setting, several blood tests can be indicative of a cytokine storm. These include determination of serum ferritin level (indirect association with IL-6), c-reactive protein (CRP), fibrinogen, serum amyloid a (SAA), Lactate dehydrogenase, procalcitonin. These tests are done at high complexity laboratories and require hours to days from specimen collection to result, during which time, in the absence of treatment with general immune suppressors or targeted treatment with anti-cytokine therapeutics, severe organ damage will continue.

The hyper-inflammatory response induced by severe SARS-CoV-2 is a major cause of severe disease and death. High serum level of IL-6, IL-8, IL-18 and TNF-α levels at the time of hospitalization have been shown to be strong and independent predictors of patient survival. The levels of several cytokines involved in COVID-19 cytokine storm syndrome in comparison to their levels in CART-induces cytokine storm is reported in https://doi.org/10.1038/s41591-020-1051-9.

The COVID-19-related cytokine responses have also been shown to be different from the cytokine storm associated with sepsis and CAR T cells with prolonged increase of cytokine levels over days and weeks, and lack of coordination between cytokines. For example, mean IL-6 serum levels are more than three times higher in patients with severe COVID-19 than those with non-complicated COVID patients. While IL-6 levels in healthy individuals is very low ranging from non-detectable to ˜5 pg/ml. IL-6 levels in COVID patients ranges from low pg/ml to as high is over 32000 pg/ml with a median of about 100 pg/ml. Similarly, IL-18 levels in COVID patients are significantly higher than those of healthy individuals with serum IL-18 level above the cut off value of 576 pg/mL on admission associated with greater than 11-fold increased risk of ICU admission.

In some embodiments, timely determination of the levels of cytokines can guide the clinicians as to the window of opportunity for specific anti-cytokine treatments, that have been received Emergency Use Authorization (EUA) or approval from FDA based on the levels and types of the cytokines that are elevated in COVID patients. This rational treatment is particularly important due to severe side effects that are associated with the use of these agents.

The cytokine storm can be particularly problematic in case of COVD patients as hospitals are overwhelmed during surges and peak COVID cases and doctors advise symptom control at home for many COVID patients. As discussed in more detail below, some embodiments of the present teachings allow the detection of a cytokine storm (or an onset of the cytokine storm), thus providing additional information that can help healthcare providers advise their patients. In other words, some embodiments of the present teachings that can detect the onset of a cytokine storm can help healthcare providers identify high-risk patients that will most benefit from rapid hospitalization and treatment management.

In use, a sample of a subject's blood can be collected, e.g., about 100 microliters of blood can be collected via a finger prick, and the blood sample can be introduced, processed to plasma/serum and mixed with a processing buffer, into the sensing modules of the above system 3000 according to the present teachings, which is configured for detection of cytokines/chemokines/inflammatory biomarkers.

The impedance signals generated by the sensing modules can be collected and analyzed by an analyzer to indicate the onset or occurrence of a cytokine storm. The analyzer can be implemented in any of software, firmware and/or hardware in a manner known in the art and as informed by the present teachings.

By way of example, based on 23 studies reported in PMID: 33058143, PMCID: PMC7646004. DOI: 10.1111/eci.13429, mean cytokine levels were significantly higher (IL-6: MD, 19.55 pg/mL; CI, 14.80, 24.30; IL-8: MD, 19.18 pg/mL; CI, 2.94, 35.43; IL-10: MD, 3.66 pg/mL; CI, 2.41, 4.92; IL-2R: MD, 521.36 U/mL; CI, 87.15, 955.57; and TNF-alpha: MD, 1.11 pg/mL; CI, 0.07, 2.15) and T-lymphocyte levels were significantly lower (CD4+ T cells: MD, −165.28 cells/p L; CI, −207.58, −122.97; CD8+ T cells: MD, −106.51 cells/μL; CI, −128.59, −84.43) among severe cases as compared to non-severe ones. There was heterogeneity across studies due to small sample sizes and nonuniformity in outcome assessment and varied definitions of disease severity. Additionally, several large studies demonstrated that IL-6 levels of greater than 80 pg/mL, are laboratory predictor of respiratory failure and death.

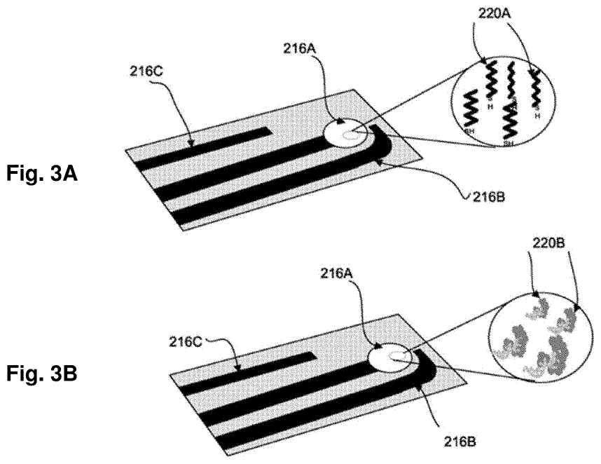

More specifically, with reference to FIGS. 3A, and 3B, in some embodiments, an electrochemical analysis cell 216 (herein also referred to as a potentiostat) suitable for use in the practice of the present teachings can include a working electrode 216a (herein also referred to as a sensing electrode), a counter electrode 216b, and a reference electrode 216c. A plurality of biorecognition elements (herein also referred to as affinity binding elements, e.g., oligonucleotides 220a or aptamers 220b in this embodiment) are coupled to the working electrode. In some embodiments, the aptamers 220b can be of the same type and can specifically bind to an epitope of a protein associated with an inflammatory target biomarker. In other embodiments, the aptamers 220b can be of different types so that they bind to different inflammatory target biomarker proteins, e.g., some of the aptamers can specifically bind to IL-6 while some of the other aptamers can specifically bind to IL-8 and/or TNF-α.

In some embodiments, the oligonucleotides coupled to the working electrode can have the same nucleotide sequence and hence can detect a single RNA or DNA segment of a genetic component associated with the target biomarker. In other embodiments, the oligonucleotides can include oligonucleotides with different nucleotide sequences so as to detect different RNA and/or DNA segments of genes coding for different target biomarkers.

Each of the electrochemical analysis cells functions by maintaining the electrical potential of the working electrode at a constant level relative to that of the reference electrode by adjusting the electrical current flowing through the counter electrode. The coupling of an inflammatory target biomarker and/or an RNA/DNA segment of a gene coding for that biomarker to an aptamer or oligonucleotide coupled to the working electrode results in a change in the current flowing through the counter electrode, thereby generating a detection signal. In other embodiments, one or more of the electrochemical cells can be implemented as two electrodes having a plurality of interdigitated fingers. In such embodiments, the electrochemical cell does not include a reference electrode.

As noted above, the aptamers used to functionalize an electrochemical cell can be selected so as to exhibit specific binding to an inflammatory target biomarker. In some other embodiments, the aptamers can be of different types, where each aptamer type exhibits specific binding to a different inflammatory target biomarker.

A sensor according to some embodiments can include, in addition to one or more sensing units that are configured to detect one or more proteins associated with anti-inflammatory target biomarker, one or more sensing units that are configured to detect one or more RNA or DNA segments associated with a gene coding for that inflammatory target biomarker. In some such embodiments, the nucleotide sequence of the oligonucleotide used to functionalize the electrochemical cell can be complementary to the nucleotide sequence of an RNA or DNA segment of the target genes.

In some other embodiments, a plurality of oligonucleotides are attached to the working electrode, where the oligonucleotides exhibit oligonucleotide sequences that are complementary to different RNA or DNA segments of the target biomarker gene (i.e., a gene coding for the an inflammatory target biomarker). By way of example, in some implementations, some of the oligonucleotides have a nucleotide sequence that is complementary to the nucleotide sequence of an RNA or DNA segment gene coding for an inflammatory target biomarker, and some of the oligonucleotides have a nucleotide sequence that is complementary to the nucleotide sequence of another RNA or DNA segment of the biomarker gene.

A variety of techniques can be employed for coupling aptamers and oligonucleotides to the working electrode of an electrochemical analysis cell. For example, in some embodiments in which the working electrode of an electrochemical analysis cell is formed of gold, the 5′ terminal end of an aptamer exhibiting specific binding to a protein biomarker or an oligonucleotide exhibiting a complementary sequence relative to the RNA and/or DNA sequence associated with a target biomarker gene can be modified with a thiol SS-C6 group to enable thiol-gold binding to the gold surface of the working electrode. The modified aptamer or oligonucleotide can then be dissolved in a tris-HCl (TE) buffer (e.g., a 1 μM buffer) to generate a mixture that can be used to functionalize the working electrode. For example, the electrode surface can be coated with the mixture via drop coating. The coated electrode can then be incubated, e.g., for 30 minutes, before rinsing it with nuclease free water and drying it under a stream of inert argon gas to remove unbound aptamers and/or oligonucleotides.

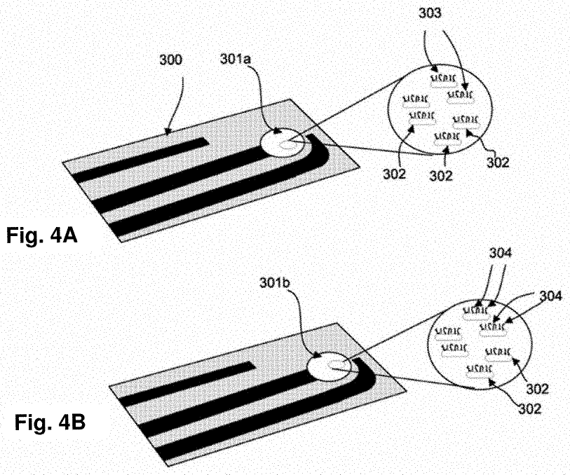

As shown schematically in FIG. 4A, in some embodiments, the working electrode 301a of an electrochemical analysis cell 300 can be functionalized with a plurality of carbon nanotubes 302 to which a plurality of aptamer probes 303 are coupled. For example, the working electrode of a sensing unit that is configured to detect a target protein of interest (a target biomarker) can be functionalized with a plurality of carbon nanotubes that are themselves functionalized with one or more aptamers that exhibit specific binding to that target protein. Further, as shown schematically in FIG. 4B, the working electrode 301b of a another electrochemical analysis cell 300′ that is configured to detect one or more RNA and/or DNA segments of a target biomarker gene can be functionalized with a plurality of carbon nanotubes 302 that are themselves functionalized with one or more oligonucleotides 304 that have a sequence complementary to the nucleotide sequence(s) of the DNA and/or RNA sequence. Once a target protein and/or a target RNA or DNA segment binds to the aptamer or the oligonucleotide, the electrochemical cell can generate a detection signal, e.g., via a change in the native fluorescence of the underlying carbon nanotubes.

The functionalization of carbon nanotubes with one or more oligonucleotide probes and/or one or more nucleotide aptamer probes can be achieved using a variety of techniques known in the art. Some such methods rely on the formation of covalent bonds between the aptamer and oligonucleotide probes and the carbon nanotubes. For example, in some such embodiments, the carbon nanotubes can be carboxylated using known techniques and covalent bonds can be formed between the COOH groups of the carboxylated carbon nanotubes and the amine groups of the probes. In other embodiments, the carbon nanotubes can be functionalized using noncovalent via π-π interactions. For example, single-stranded DNA and RNA molecules with aromatic bases can be immobilized on the surfaces of carbon nanotubes via such interactions. Further details regarding various techniques for coupling aptamer and oligonucleotide probes to the surfaces of carbon nanotubes can be found, e.g., in an article entitled “Aptamer-functionalized carbon nanomaterials electrochemical sensors for detecting caner relevant biomolecules,” published in Carbon 129 (2008) 380-395, which is herein incorporated by reference in its entirety.

The functionalization of gold electrodes with oligonucleotides, aptamers or with carbon nanotubes (CNTs) that are functionalized with oligonucleotides or aptamers can be achieved in some embodiments via direct self-assembly or via Chitosan.

For example, CNTs can be carboxylated via acid treatments in a manner known in the art. The negatively charged carboxyl groups on CNTs allow two approaches for direct self-assembly and immobilization of CNTs on electrode surfaces: one is the covalent bonding of CNTs to cysteamine self-assembled monolayer (SAM) modified gold electrodes via the reaction of carboxyl groups on CNTs and amino groups on cysteamine SAM in the presence of coupling reagents; the other is the attachment of CNTs to electrode surfaces via the electrostatic interactions between negatively charged carboxyl groups on CNTs and the positively charged species on the electrode surfaces. The self-assembly of CNTs on a gold electrode has been reported. In one such approach, as-grown carbon nanotubes were cut into short pipes and thiol-derivatized at their open ends by chemical methods. The ordered assembly of SWCNTs was then achieved by their spontaneous chemical adsorption to gold via Au—S bonds. Such an approach can result in the formation of a self-assembled monolayer of CNTs on gold with a perpendicular orientation. In contrast to the covalent methods for attaching CNTs to cysteamine modified gold electrodes, the attachment of acid-treated SWCNTs to cysteamine modified gold electrodes via electrostatic adsorption has also been reported. In addition, methods for preparing aligned CNT arrays on the surface of ordinary pyrolytic graphite (PG) electrodes from randomly dispersed CNTs by using a Nafion solution is also known.

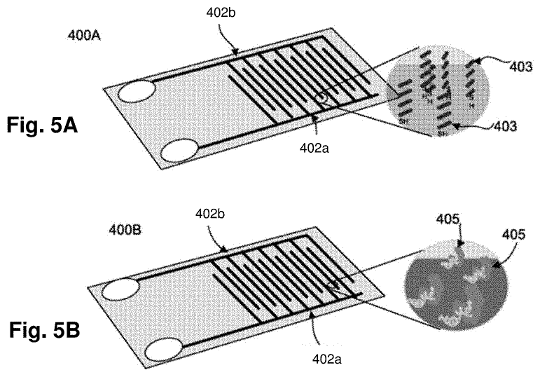

As shown schematically in FIGS. 5A and 5B, in some embodiments, an electrochemical analysis cell 400a/400b according to the present teachings includes two interdigitated electrodes 402a/402b, each of which has a plurality of conductive fingers, where the fingers of the two electrodes are interleaved to form an interdigitated structure. One of the electrodes can function as the working electrode of the electrochemical analysis cell and the other electrode can function as the counter electrode. Without being limited to any particular theory, the close proximity of the fingers of the two electrodes (e.g., two adjacent fingers of the two electrodes can be separated by a distance, for example, in a range of about 5 microns to about 1 mm, though other distances can also be employed) can obviate the need for a reference electrode. The working electrode (i.e., the fingers of the working electrode) can be functionalized directly or via a plurality of carbon nanotubes in a manner discussed herein to allow detection of one or more proteins or one or more RNA/DNA segments of a target genetic component associated with the biomarker. For example, FIG. 5A schematically depicts a plurality of oligonucleotide probes 403 that are coupled to the surface of the working electrode and FIG. 5B schematically depicts a plurality of aptamer probes 405 that are coupled to the surface of the working electrode in a manner discussed herein.

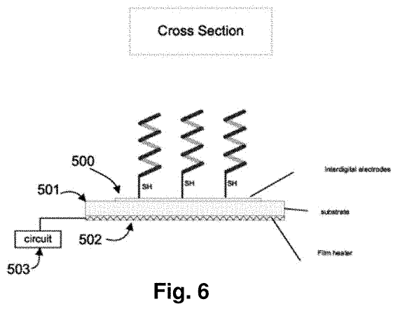

In some embodiments, a heating element can be incorporated into an electrochemical cell according to the present teachings for heating, for example, the working electrode and hence the aptamer and/or oligonucleotide probes 405 coupled to the working electrode. For example, FIG. 6 schematically depicts a working electrode 500 of such an electrochemical cell that is deposited on an underlying substrate 501 (e.g., a glass, plastic, or other suitable substrates). A heating element 502 in the form of a resistive element is disposed below the substrate 501 to heat the substrate and the working electrode. In this embodiment, an electrical circuit 503 can apply a current to the resistive film to cause heating thereof. Such an electrical circuit can be implemented in a manner known in the art, e.g., by implementing a current source. By way of example, the heating element can be used to heat the working electrode to a temperature in a range of about 60° C. to about 65° C. Without being limited to any particular theory, in some embodiments, such heating can advantageously disentangle those aptamers and/or oligonucleotides that have been entangled, e.g., due to close proximity and the natural molecular motion of the probes, thereby allowing a more facile interaction of the probes with target proteins and/or RNA/DNA segments. A control unit (not shown) can control the operation of the heating element, for example, for activating the heating element for a selected time period followed by deactivating the heating element.

In some embodiments, commercial electrochemical sensors can be obtained and modified in accordance with the present teachings, e.g., to functionalize one of their electrodes with aptamer or oligonucleotide probes. By way of example, electrochemical sensors marketed by Metrohm DropSens of Oviedo, Spain can be employed in some implementation of a sensor according to the present teachings.

In some embodiments, a sensing unit configured via functionalization with one or more oligonucleotide probes to detect one or more RNA/DNA sequences associated with a target biomarker can generate a detection signal even in absence of a complete complementarity between the probe sequence and a target RNA/DNA segment. For example, in some embodiments, such a sensing unit can generate a detection signal when a single nucleotide polymorphism (SNP) is present in the target RNA/DNA segment of interest.

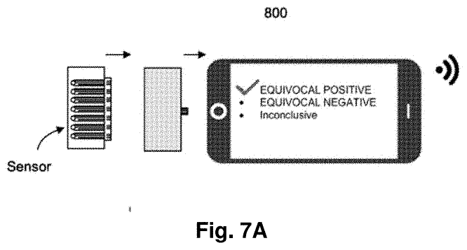

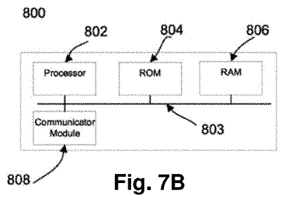

FIG. 7A schematically depicts an analyzer 800 according to an embodiment, which can receive data from a sensor according to the present teachings and process that data to indicate whether a target biomarker is present in a sample. For example, when at least one protein-detecting sensing unit of a sensor indicates that presence of at least one protein biomarker in the sample, and at least one RNA/DNA detecting sensing unit of the sensor indicates the presence of at least one RNA and/or DNA segment associated with the target biomarker, the analyzer can provide an indication, e.g., via textual and/or graphical means, that the target biomarker is present in the sample. The analyzer can also employ other criteria, e.g., more stringent criteria, for indicating that a target biomarker is present in a sample under study. For example, the analyzer may be configured to indicate the presence of a biomarker in the sample when both protein and genetic signals are detected.

By way of example, the analyzer 800 can indicate whether the test result is positive (i.e., the target biomarker is present in the sample), it is negative (i.e., the target biomarker is not present in the sample), or the test result is inconclusive. The negative indication can be provided when no protein or genetic component signals is detected. The inconclusive indication may be provided, for example, when only a protein or only a genetic component signal is detected. A positive indication may be provided when both a protein and a genetic component signal are detected.

The analyzer 800 can be implemented in hardware, software and/or firmware in a manner known in the art informed by the present teachings. For example, FIG. 7B schematically depicts such an implementation of the analyzer, which includes a processor 802, at least one random access memory module (RAM) 804, at least one permanent memory module (ROM) 806, a communication module 808 (e.g., a wireless communication module using any of the known protocols). The processor 802 can communicate with the other components of the analyzer via a communication bus 803. The instructions for analyzing the data received from a sensor can be stored in the ROM module 804 and be transferred into the RAM module 806 by processor for execution.

In some embodiments, at least one sensing unit of a sensor according to the present teachings is configured to provide a negative control. By way of example, one sensing unit can be functionalized with aptamers for detecting actin protein.

In some embodiments, a sensing electrode of an electrochemical sensing unit according to one aspect of the present teachings can be functionalized with a plurality of nanoparticles, which are in turn functionalized with an affinity binding element that is configured to exhibit specific binding to a target biomarker. By way of example, in some embodiments, nanoparticles can be substantially spherical with a diameter in a range of about 5 nm to about 100 nm, e.g., in a range of about 10 nm to about 50 nm, or in a range of about 20 nm to about 30 nm, though other sizes can also be employed. By way of example, the nanoparticles can be formed of gold.

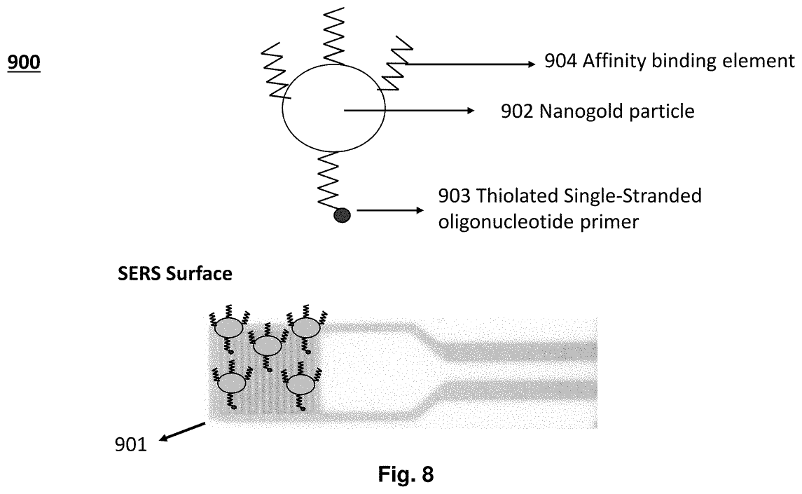

FIG. 8 schematically depicts an example of such a functionalized sensing electrode 900 that includes an underlying gold layer 901 and a plurality of gold nanoparticles 902 that are distributed over the gold layer 901.

The gold nanoparticles 902 can in turn be functionalized with a plurality of affinity binding elements 904. By way of example, the gold nanoparticles can be functionalized with affinity binding elements that exhibit specific binding to a target protein or a target genetic component (e.g., an RNA segment) of an inflammatory target biomarker. In some embodiments, the affinity binding elements 904 can also include at least one ligand that can facilitate anchoring, e.g., via covalent bonds, the gold nanoparticles to the underlying gold layer. An example of such coupling ligand can be a thiol group, e.g., a cysteine group. In some embodiments, an affinity binding element of interest can be thiolated to allow its coupling to the underlying gold layer.

A variety of techniques for the synthesis of gold nanoparticles (AuNPs) are known. By way of example, colloidal AuNPs can be prepared as follows: 0.5 mL of 1% (w/v) sodium citrate solution can be added to 50 mL of 0.01% (w/v) HAuCl4 boiling solution. HAuCl4 and sodium citrate aqueous solutions can be filtered through a 0.22 μm microporous membrane filter before using. The mixture can be boiled for 15 minutes and then stirred for 15 minutes after removing the heating source to produce colloidal gold nanoparticles. The mixture can be stored in a refrigerator in a dark-colored glass bottle before using.

In some embodiments, commercially available gold nanoparticles, such as those marketed by Nanopartz of Loveland CO, USA can be employed. By way of example, some such gold particles can have pentahedrally-faceted profiles and can have diameters in a range of 60 nm to 100 nm with size accuracies better than 5 nm and size variances less than 10%. They exhibits peak SPRs (surface plasmon resonances) in a range of 780 nm to 980 nm.

As noted above, the gold nanoparticles can be anchored to the underlying gold surface using a variety of ligands. For example, cysteine ligands can be used to immobilize AuNPs to the underlying gold surface. In one method of functionalizing an underlying gold surface with a plurality of AuNPs, the gold surface can be cleaned, e.g., via exposure to a plasma. In other embodiments, the cleaning of the gold surface can be achieved via polishing the surface with an abrasive paper, followed by rinsing the surface with ethanol and distilled water and then drying the surface with filter paper. The cleaned gold surface can then be immersed in a cysteine aqueous solution (e.g., a 0.1 M cysteine aqueous solution) for a few hours, e.g., for 2 hours, at room temperature in darkness. The resulting modified electrode can then be rinsed thoroughly with distilled water and soaked in distilled water for 12 hours in order to remove any physically-adsorbed cysteine. The cysteine-functionalized gold surface can then be dipped into the colloidal gold solution for 24 hours at 4° C. The AuNPs self-assembled electrode can be dipped into distilled water for conservation at 4° C.

Further details regarding synthesis and functionalization of a gold surface with functionalized gold nanoparticles are provided in an article entitled “Single Layer of Gold Nanoparticles Self-Assembled on Gold Electrode as a Novel Sensor with High Electrocatalytic Activity,” published in Journal of Analytical Chemistry, 2018, Vol. 73, No. 11, pp. 1118-1127. © Pleiades Publishing, Ltd., 2018, which is herein incorporated by reference in its entirety.

In some embodiments, an affinity binding element (e.g., an aptamer or other oligonucleotides) of interest with which a sensing electrode of a detector according to the present teachings is functionalized can include ligands (e.g., thiol groups) that allow coupling the affinity binding element to the sensing electrode. In other cases, an affinity binding element can be chemically modified to include ligands (e.g., thiol groups) that would facilitate its attachment to the working electrode.

The functionalization of a sensing electrode of an electrochemical sensing unit can advantageously increase the effective surface area of the sensor, thereby enhancing the sensitivity of the sensor for the detection of an antigen of interest.

In some embodiments, a detection system according to an embodiment of the present teachings can include, in addition to one or more sensors having one or more electrochemical sensing units, at least one sensing unit that employs surface enhanced Raman spectroscopy (SERS) for the detection of a protein biomarker an RNA or a DNA segment of a gene associated with the biomarker.

In some such embodiments, a sensing electrode can function as an electrochemical sensor and can also provide a surface suitable for performing surface enhanced Raman spectroscopy. By way of example, the gold nanoparticle functionalized gold layer depicted schematically in FIG. 8 can provide such dual sensing functionality.

For example, in some such embodiments, the electrode 900 can be used in a manner discussed above as an electrochemical sensing unit to generate an electrical signal in response to the coupling of an antigen of interest, when present in a sample under investigation, to the affinity binding element coupled to the electrode surface. The electrode 900 can also be used as a SERS surface to allow interrogation of the sample via Raman spectroscopy. In some embodiments, the gold nanoparticles can have sizes, e.g., in a range of about 6 nm to about 100 nm, though other sizes can also be employed.

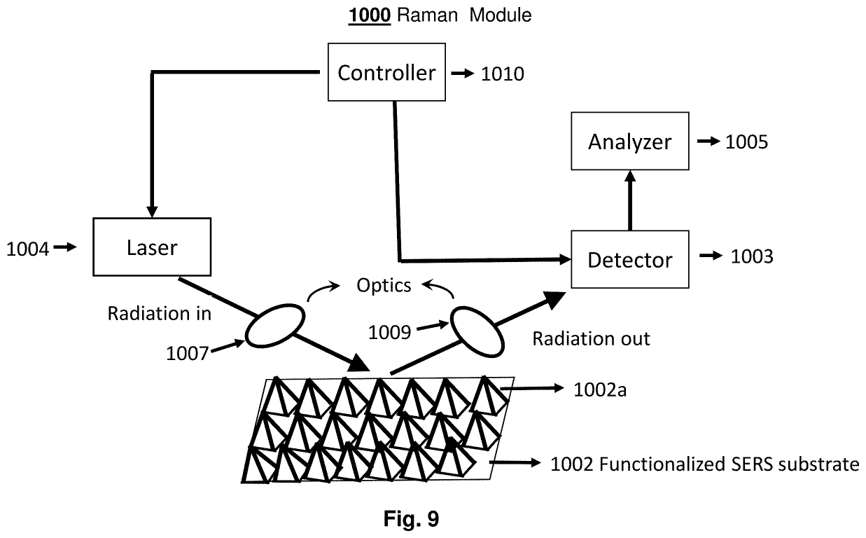

In some embodiments, a system according to the present teachings can include a SERS module that is separate from the electrochemical sensing modules. In other words, in some such embodiments, the SERS module can be employed only for obtaining SERS data. By way of example and with reference to FIG. 9 such a SERS module 1000 can include a SERS surface 1002 that includes a plurality of metalized protrusions/corrugations 1002a.

The surface 1002 can be functionalized with an affinity binding element that can provide specific binding to a target biomarker of interest.

The SERS module can further include a laser source 1004 that can provide radiation for exciting one or more Raman active transitions of either the affinity binding element and/or the respective protein target biomarker of interest or a genetic component associated therewith. In this embodiment, the laser radiation is directed via one or more optics 1007 onto the functionalized SERS surface and the Raman scattered radiation can be detected via a detector 1003. In this embodiment, one or more optics 1009 are disposed in front of the detector for focusing the Raman-scattered radiation onto the detector. The detector generates detection signal(s) in response to the detection of the Raman-scattered radiation, which are received by an analyzer 1005 that is in communication with the detector 1003 to receive the detection signal(s) generated by the detector.

The analyzer 1005 is configured to process the received Raman-scattered detection signals to identify and analyze the Stokes and/or anti-Stokes Raman peaks to determine whether the detected Raman signal indicates the presence of the target inflammatory biomarker in a sample under investigation. In this embodiment, a controller 1010 controls the operation of the laser and detector. For example, among other functions, it can provide triggers for activation of the laser and the detector.

The use of both electrochemical as well as Raman data can increase the reliability of a sensor according to such embodiments, and lower the rate of false positive signals. For example, the use of different detection modalities (i.e., electrochemical and optical) can help enhance the sensor's reliability by providing data in at least two detection channels that rely on different physical/chemical processes for the detection of a biomarker of interest.

Those having ordinary skill in the art will appreciate that various changes can be made to the above embodiments without departing from the scope of the invention.

What is claimed is:

1. A system for detecting inflammation, comprising
at least one sensor comprising:
at least one port for receiving a biological sample,
at least one electrochemical cell in fluid communication with said at least one port for receiving said biological sample, said electrochemical cell comprising at least two conductive electrodes, where at least one of said electrodes is functionalized with at least one molecular recognition probe exhibiting specific binding to at least one inflammatory target biomarker, inflammation biomarker or genetic component associated with an inflammatory target biomarker,
circuitry for detecting any of a redox current flowing through said at least one electrode and/or an electrical impedance across said electrodes in response to interaction of said functionalized electrode with said sample and generating detection signals in response to said detection.

2. The system of claim 1, wherein said at least one electrochemical cell comprises a plurality of electrochemical cells each of which is configured for detection of a different biomarker.

3. The system of claim 1, wherein said probe comprises any of: an aptamer, SOMAmer, an antibody, a raptomer, a megastar or other antibody or non-antibody, and/or a synthetic affinity binding agent specific to a target of interest.

4. The system of claim 1, further comprising an analyzer in communication with said circuitry for receiving said signals and processing the signals to identify an onset and/or an occurrence of a cytokine storm.

5. The system of claim 1, wherein said at least one electrochemical cell comprises a first electrochemical cell functionalized with a probe exhibiting specific binding to said inflammation biomarker and a second electrochemical cell functionalized with a probe exhibiting specific binding to said genetic component associated with the inflammation biomarker.

6. The system of claim 5, further comprising an analyzer configured to receive the detection signals generated by said first and second electrochemical cells and process said detection signals to determine whether the inflammation biomarker is present in the biological sample.

7. The system of claim 6, wherein said analyzer indicates presence of the inflammation biomarker in the biological sample when both of the detection signals generated by the first and second electrochemical cells are positive signals.

8. The system of claim 6, wherein said analyzer indicates absence of the inflammation biomarker in the biological sample when both of the detection signals generated by the first and second electrochemical cells are negative signals.

9. The system of claim 6, wherein said analyzer indicates a non-conclusive result when one of said detections signals generated by the first and the second electrochemical cells is positive and the other detection signal is negative.

10. The system of claim 1, wherein said inflammation biomarker comprises any of a cytokine and a chemokine.

11. The system of claim 1, wherein said inflammation biomarker comprises any of interferon-γ, interleukin-6, interleukin-8, interleukin-10, interleukin-18 and soluble interleukin-2 receptor alpha, CXCL9 and CXCL10.

12. The system of claim 10, wherein said chemokine is induced by interferon-γ.

13. The system of claim 10, wherein said biological sample comprises any of: blood, urine, or other liquid biopsy or exosomes prepared from any liquid biopsy.

14. A method for detecting an inflammation biomarker in a biological sample, comprising:
introducing the biological sample into an electrochemical cell of a sensor having at least one working electrode and a reference electrode, wherein the working electrode is functionalized with at least one molecular recognition probe exhibiting specific binding to said inflammation biomarker, or a genetic component coding for that inflammation biomarker, and
measuring a redox current flowing through said working electrode or an electrical impedance across said working electrode and said reference electrode in response to interaction of said functionalized electrode with the sample so as to generate detection signals.

15. The method of claim 14, further comprising processing said detection signals to determine whether said inflammation biomarker is present in said biological sample at a concentration level above a limit-of-detection of said sensor.

16. The method of claim 15, wherein said inflammation biomarker comprises any of a cytokine and a chemokine.

17. The method of claim 16, wherein said inflammation biomarker comprises any of interferon-γ, interleukin-6, interleukin-8, interleukin-10, interleukin-18 and soluble interleukin-2 receptor alpha, CXCL9 and CXCL10.

18. The method of claim 16, wherein said chemokine is induced by interferon-γ.

19. The method of claim 15, wherein said biological sample comprises any of blood and urine.


SEQUENCE LISTING (16 sequences)

SEQ ID NO: 1 - RNA, 31 nt, Artificial Sequence
  Description of Artificial Sequence Synthetic oligonucleotide
ggcaguuugguauuaacacguuaagucgugg

SEQ ID NO: 2 - RNA, 19 nt, Artificial Sequence
  Description of Artificial Sequence Synthetic oligonucleotide
ggggaggcuguggugaggg

SEQ ID NO: 3 - RNA, 50 nt, Artificial Sequence
  Description of Artificial Sequence Synthetic oligonucleotide
guaaguaguguaggcugugggaguuauagggguggauguggaguggggug

SEQ ID NO: 4 - RNA, 34 nt, Artificial Sequence
  Description of Artificial Sequence Synthetic oligonucleotide
gggagaacugugggaguggaggguggaugguucu

SEQ ID NO: 5 - RNA, 22 nt, Artificial Sequence
  Description of Artificial Sequence Synthetic oligonucleotide
cgugaguuauggguuagagucg

SEQ ID NO: 6 - DNA, 30 bp, Artificial Sequence
  Description of Artificial Sequence Synthetic oligonucleotide
gaagtatagcgcaataaactcacctcccac

SEQ ID NO: 7 - DNA, 41 bp, Artificial Sequence
  Description of Artificial Sequence Synthetic oligonucleotide
gcgccactacaggggagctgccattcgaataggtgggccgc

SEQ ID NO: 8 - DNA, 25 bp, Artificial Sequence
  Description of Artificial Sequence Synthetic oligonucleotide
tggtggatggcgcagtcggcgacaa

SEQ ID NO: 9 - RNA, 28 nt, Artificial Sequence
  Description of Artificial Sequence Synthetic oligonucleotide
ggaguaucugaugacaauucggagcucc

SEQ ID NO: 10 - DNA, 49 bp, Artificial Sequence
  Description of Artificial Sequence Synthetic oligonucleotide
tccgatcggtatatccgtcggattttttttttggtcactgcatgtgacc

SEQ ID NO: 11 - RNA, 35 nt, Artificial Sequence
  Description of Artificial Sequence Synthetic oligonucleotide
gggggcuuaucauuccauuuaguguuaugauaacc

SEQ ID NO: 12 - RNA, 104 nt, Artificial Sequence
  Description of Artificial Sequence Synthetic polynucleotide
gggcgaauucgggacuucgauccguaguacccaccaggcauacaccagcacgcggagccaaggaaaaauaguaaacuagcacucagugcucguaugcggaagcu

SEQ ID NO: 13 - DNA, 71 bp, Artificial Sequence
  Description of Artificial Sequence Synthetic oligonucleotide
ggcaggaagacaaacacgatggggggtatgatttgatgtggttgttgcatgatcgtggtctgtggtgctgt

SEQ ID NO: 14 - DNA, 52 bp, Artificial Sequence
  Description of Artificial Sequence Synthetic oligonucleotide
cgaaggggattcgaggggtgattgcgtgctccatttggtgtttttttttttt

SEQ ID NO: 15 - DNA, 79 bp, Artificial Sequence
  Description of Artificial Sequence Synthetic oligonucleotide
agcagcacagaggtcagatgccccgcgggtcggcttgccgttccgttcggcgcttcccccctatgcgtgctaccgtgaa

SEQ ID NO: 16 - DNA, 79 bp, Artificial Sequence
  Description of Artificial Sequence Synthetic oligonucleotide
tcctctgactgtaaccacgaaggtgtcggccttagtaaggctacagccaagggaacgtagcataggtagtccagaagcc